(19) United States
(12) Patent Application Publication
(10) Pub. No.: US 2010/0297766 A1
(43) Pub. Date: Nov. 25, 2010

(54) Method of Removing Undesired Tissue from Connective Tissue

(76) Inventors: Hai-Qing Xian, Austin, TX (US); Hali Wang, The Hills, TX (US); Hui Liu, Austin, TX (US); Jian Q. Yao, Austin, TX (US); Jizong Gao, Cedar Park, TX (US)

Correspondence Address: HOWREY LLP-HN, C/O IP DOCKETING DEPARTMENT, 2941 FAIRVIEW PARK DRIVE, SUITE 200, FALLS CHURCH, VA, 22042-7195, US

(21) Appl. No.: 12/864,207

(22) PCT Filed: Feb. 3, 2009
(86) PCT No.: PCT/US09/32961

§ 371 Date: Jul. 22, 2010

Related U.S. Application Data
(60) Provisional application No. 61/026,012, filed on Feb. 4, 2008.

(51) Int. Cl. C12N 5/02

(52) U.S. Cl. 435/379

(57) ABSTRACT

A method of removing undesired tissue from a connective tissue is provided that includes stretching the connective tissue and peeling away the undesired tissue. Some methods include soaking the connective tissue in a solution prior to and/or subsequent to stretching and peeling away the undesired tissue. The solution may include a detergent, an enzyme, a salt solution, an organic solvent, and combinations thereof.

### FIELD OF THE INVENTION

The present invention generally relates to the preparation of connective tissue for transplant, and more specifically relates to methods for removal of undesired tissue from connective tissue.

### BACKGROUND

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Connective tissue, examples of which include tendons, ligaments, and menisci, can become damaged and require surgical repair. Such surgical repair may include grafting a replacement connective tissue, such as tendon or ligament, to the subject. While some replacement connective tissue may come from the body of the injured subject (an autograft), other replacement connective tissue comes from a donor. Donated connective tissue may be from the same species (allograft) or different species (xenograft) as the injured subject (i.e., recipient or host).

A layer of undesired tissue covers some connective tissue. For example, such undesired tissue may include a fatty tissue layer that is found in xenograft connective tissue (relative to humans), such as certain porcine tendons. The undesired tissue can potentially cause an inflammatory and/or immune reaction if transplanted into a recipient, thus making removal of the undesired tissue prior to grafting the connective tissue advantageous. Conventional methods of removing undesired tissue from connective tissue involve cutting the undesired tissue away from the connective tissue using a scalpel or other sharp instrument. However, this method of removing undesired tissue is limited because (1) the undesired tissue may not be evenly and completely removed, (2) there is significant risk of damaging the connective tissue with the sharp instrument, and (3) it requires a great amount of skill, time, and effort.

In view of the drawbacks of conventional methods of undesired tissue removal, new and/or improved methods of undesired tissue removal are desirable.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

One aspect of the present invention provides a method of removing undesired tissue from connective tissue without one or more of the limitations of the conventional methods described above. To that end, the method generally includes stretching connective tissue, such as, for example, a tendon, ligament or meniscus, and peeling the undesired tissue from the stretched connective tissue. More specifically, an intermediate portion of the connective tissue, which extends between first and second opposing ends of the connective tissue, is stretched. The connective tissue may be excised from a donor and/or soaked in a solution prior to stretching the connective tissue and peeling away the undesired tissue. Once the connective tissue has been stretched, the undesired tissue may be cut along the width of the connective tissue, to provide a leading tissue edge that may be gripped. This leading tissue edge is then gripped and force applied to peel the undesired tissue away from the connective tissue. Should any remnants of the undesired tissue remain, these may be removed with a cleaning solution (including detergents, enzymes, organic solvents, etc.) or by mechanical means such as scrubbing with a brush. The method results in connective tissue that has substantially all of the undesired tissue evenly removed, such that the connective tissue is ready for further processing to eliminate immunological and inflammatory responses of the recipient. Further, the undesired tissue is removed with minimal risk of damaging the connective tissue, and with reduced or minimal skill, time, and effort. By virtue of the foregoing, there is thus provided a method of removing undesired tissue from connective tissue without one or more of the limitations of conventional methods.

Various features discussed below in relation to one or more of the exemplary embodiments may be incorporated into the above-described aspect of the present invention alone or in any combination. Again, the brief summary presented above is intended only to familiarize the reader with certain aspects and contexts of the present invention without limitation to the claimed subject matter.

### BRIEF DESCRIPTION OF THE DRAWINGS

Various features, aspects, and advantages of the present invention will become better understood when the following detailed description is read with reference to the accompanying figures in which like characters represent like parts throughout the figures, wherein:

FIG. 1A is a perspective view of a tendon excised from a subject.

FIG. 1B is a cross-sectional view of the tendon of FIG. 1A, taken along line 1B-1B of FIG. 1A.

FIG. 2 is a perspective view of an exemplary embodiment in which the tendon is soaked in a solution.

FIG. 3 is a perspective view of a stretched tendon.

FIG. 4 is a perspective view of a stretched tendon having a portion of the tissue peeled away from the tendon.

FIG. 5 is a perspective view of the tendon with substantially all of the undesired tissue evenly removed.

### DETAIL DESCRIPTION

One or more specific embodiments of the present invention will be described below. In an effort to provide a concise description of these embodiments, all features of an actual implementation may not be described in the specification. It should be appreciated that in the development of any such actual implementation numerous implementation-specific decisions must be made to achieve the developers' specific goals, which may vary from one implementation to another. Moreover, it should be appreciated that such a development effort might be complex and time consuming, but would nevertheless be a routine undertaking for those of ordinary skill having the benefit of this disclosure.

When introducing elements of the present invention (e.g., the exemplary embodiments(s) thereof), the articles “a”, “an”, “the” and “said” are intended to mean that there are one or more of the elements. The terms “comprising”, “including” and “having” are intended to be inclusive and mean that there may be additional elements other than the listed elements.

As described in the Background section, connective tissues, for example, include tendons, ligaments, and menisci. Tendons and ligaments are tough, inelastic, but flexible bands of connective tissue found in the body that attach muscle to bone or bone to bone, respectively. Tendons generally consist of numerous parallel collagen fibers that serve to attach muscles to bones. Ligaments generally consist of fibrous connective tissue that links two bones together at a joint. Some ligaments also provide support for organs by attaching them to surrounding muscle and bone. In addition, some tendons may form connections between bones, such as for example, the patellar tendon, which connects the patella to the tibia. Menisci are discs of connective tissue found in the joints between bones. Menisci generally consist of cartilage that divides the cavity of a joint.

One aspect of the present invention provides a method of removing tissue from a connective tissue, such as, for example, a tendon, ligament, or meniscus, without one or more of the limitations of the conventional methods described above. To that end, and referring to the Figures, the method generally includes stretching a connective tissue and peeling the undesired tissue (10) from the stretched connective tissue. More specifically, an intermediate portion (16) of the connective tissue (i.e., tendon (12), ligament, or meniscus), which extends between a first opposing end (20) and a second opposing end (22) of the connective tissue, is stretched. The connective tissue may be excised from a donor and/or soaked in a solution prior to or during stretching the connective tissue and peeling away the undesired tissue (10). The undesired tissue (10) may be cut along the width of the connective tissue, to provide a leading tissue edge (34) that may be gripped either prior to or after the connective tissue has been stretched. This leading tissue edge (34) is gripped and force applied to peel the undesired tissue (10) away from the connective tissue. Should any remnants of undesired tissue (10) remain, these may be removed with a cleaning solution (including detergents, enzymes, organic solvents, etc.) and/or with mechanical means such as, for example, scrubbing with a brush. The method results in a connective tissue that has substantially all of the undesired tissue (10) evenly removed. Further, the undesired tissue (10) is removed with minimal risk of damaging the connective tissue, and with minimal skill, time, and effort.

The methods of the present invention may be used with any connective tissue, such as, for example, a tendon (12), ligament, or meniscus, found in a subject that requires the removal of undesired tissue (10). For example, particular connective tissue such as tendons (12) or ligaments used in xenograft implantations may include such a layer of undesired tissue. One example is a porcine patellar tendon. Thus, in one embodiment, the connective tissue is a porcine patellar tendon.

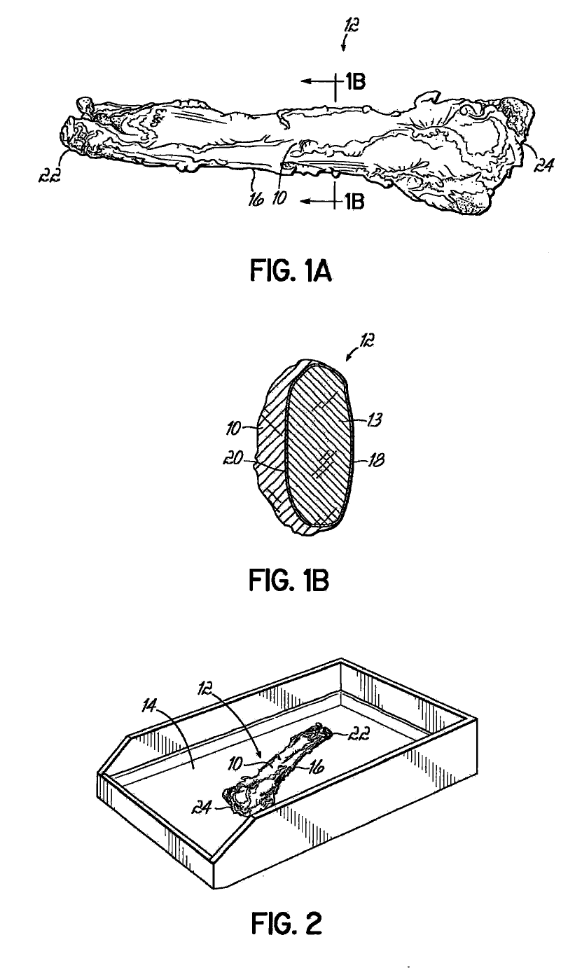

More specifically, and as illustrated in FIGS. 1A through 5, a tendon (12) used in the methods of the present invention generally has an intermediate portion (16) having an outer surface (20) extending between first (22) and second (24) opposing ends. As illustrated in FIG. 1B, tendons (12) generally have an inner portion (13) comprised of primarily connective tissue that is surrounded by an outer covering of peritendineum (18). Some tendons may have a layer of undesired tissue (10) on the outer surface (20) of the peritendineum (18). As further illustrated in FIG. 1B, the undesired tissue (10) may be found on the outer surface (20) of the peritendineum (18), such as, for example, the anterior outer surface (20) of the peritendineum (18). The undesired tissue (10) requires removal prior to implantation of the connective tissue. The undesired tissue (10) may include layers of fat, connective, or other tissue such as, for example, the layer of fat tightly attached to the porcine patellar tendon.

In one embodiment, the connective tissue used in the present invention may be excised from a subject prior to removal of the undesired tissue (10). One or both of the first and second opposing ends (22, 24) may optionally remain attached to bone, muscle, or organ tissue after excision from the subject. The attached bone, muscle, or organ tissue may provide points for securing one or both opposing ends (22, 24) of the connective tissue prior to stretching the intermediate portion (16). It is contemplated that the methods of the present invention can be performed when the connective tissue does not remain attached to bone, muscle, or organ tissue after excision. It is further contemplated that the methods of the present invention can be performed when the undesired tissue (10) is removed from the connective tissue prior to excision from the subject.

As illustrated in FIG. 2, in some embodiments, the connective tissue, such as a tendon (12), ligament, or meniscus, may be soaked in a solution (14) prior to stretching. In one embodiment, such a solution (14) may include multiple components, including, but not limited to, one or more of water, a detergent, an enzyme, a salt solution, and an organic solvent (as will be discussed in greater detail below). Soaking the connective tissue in such a solution (14) functions to loosen the tissue (10) so that it is more easily removed from the tendon (12) or ligament. In one embodiment, the connective tissue is soaked for at least about 0.5 hours. In another embodiment, the connective tissue is soaked for at least about 2 hours. In yet another embodiment, the connective tissue is soaked for at least about 8 hours. In a further embodiment, the connective tissue is soaked for at least about 12 hours. In still another embodiment, the connective tissue is soaked for at least about 24 hours. The methods of the invention are not limited to the times disclosed herein. Those of ordinary skill in the art will understand that the times can be adjusted as may be needed to practice various embodiments.

In some embodiments, as the connective tissue is soaked in the solution (14), the temperature range is carefully controlled for various purposes such as, for example, activating enzymes, solubilizing fat, and preserving the connective tissue. For example, a particular enzyme may require a particular temperature to be activated. In some embodiments the connective tissue may be soaked in the solution (14) in a temperature range of about 4° C. to about 50° C. In another embodiment the temperature of the solution (14) is in a range of about 4° C. to about 42° C. In yet another embodiment, the temperature of the solution (14) is in a range of about 20° C. to about 37° C. The methods of the invention are not limited to the temperature ranges disclosed herein. Those of ordinary skill in the art will understand that the temperature of the solution (14) can be adjusted as necessary to practice various embodiments.

As described briefly above, the solution (14) for use in the methods of the present invention may include one or more components chosen from water, a detergent, an organic solvent, an enzyme, a salt solution, and combinations thereof. Each of these components may individually, or in combination, loosen the undesired tissue (10) to make peeling away the undesired tissue (10) from the connective tissue easier. For example, water may be used alone to loosen the undesired tissue (10) from the connective tissue, or as a general solvent for the additional components. The detergents and organic solvents solubilize fat or lyse cells in the undesired tissue. The enzymes digest undesired components of connective and fat tissues and the salt solutions loosen the undesired tissue (10).

In embodiments where the solution (14) contains a detergent, any ionic or non-ionic detergent that effectively loosens the undesired tissue (10) from the connective tissue, such that the connective tissue remains viable for transplant, may be used in the present invention. Examples of suitable detergents include sodium dodecyl sulfate (“SDS”), sodium deoxycholate (“SDC”), t-Octylphenoxypolyethoxyethanol (also known as Triton® X-100), polyoxyethylene-4-lauryl ether (also known as Brij-35®, octylphenol-ethyleneoxide (also known as NP-40®), poly(ethylene glycol)p-nonyl-phenyl-ether (also known as nonoxynol-9®), Allowash® (a proprietary combination of Brij-35®, NP-40®, and Nonoxynol-9®), n-lauroyl sarcosinate (“NLS”), polyoxyethylenesorbitan (also known as Tween® 20), and combinations thereof. Detergents are used in the solution (14) of some embodiments of the invention at concentrations sufficient to loosen the undesired tissue (10) from the connective tissue. By way of example, SDS, SDC, Triton® X-100, or NLS may be in solution (14) in a range of about 0.01% wt/vol. to about 10% wt/vol. and Allowash® may be in solution (14) in a range of about 0.001× to about 10×. However, these detergents and ranges are merely exemplary, and those of ordinary skill in the art will understand that the detergents and concentrations can be adjusted as necessary to practice various embodiments.

In embodiments where the solution (14) contains an enzyme, any enzyme that may assist in removing undesired tissue (10) from the connective tissue, such that the connective tissue remains viable for transplant, may be used. Examples of suitable enzymes include lipase A, lipase B, trypsin, chymotrypsin, and combinations thereof. It is contemplated that other enzymes may be utilized in the present invention, such as, for example, esterase. The enzymes are present in the solutions (14) used in the methods of this invention at concentration sufficient to loosen the undesired tissue (10) from the connective tissue. By way of example, lipase A and lipase B may be used in the solution in a range of about 100 units/ml to about 10,000 units/ml and trypsin may used in range of about 0.025% wt/vol. to about 2.5% wt/vol. Chymotrypsin and esterase may each be used in a range of about 1 μg/ml to about 1000 μg/ml, However, these enzymes and ranges are merely exemplary, and those of ordinary skill in the art will understand that the enzymes and concentrations can be adjusted. as necessary to practice various embodiments.

In embodiments where the solution (14) contains a salt solution, any salt solution that effectively loosens the undesired tissue (10) from the connective tissue, such that the connective tissue remains viable for transplant, may be used. Examples of suitable salt solutions include phosphate buffered saline (“PBS”), Tris solution, Hepes solution, sodium chloride solutions (“NaCl solutions”), calcium chloride solutions (“CaCl₂ solutions”), and combinations thereof. The salt solutions may be hypertonic, hypotonic, or isotonic. The osmolarity of the salt solution maybe from about 0.001× to about 5× of physiological osmolarity. However, these salt solutions and ranges are merely exemplary, and those of ordinary skill in the art will understand that the salt solutions and concentrations can be adjusted as necessary to practice various embodiments.

In embodiments where the solution (14) contains an organic solvent, any organic solvent that effectively loosens the undesired tissue (10) from the connective tissue, such that the connective tissue remains viable for transplant, may be used. Examples of suitable organic solvents include tri(n-butyl)phosphate (“TBP”), ethanol, isopropanol, and combinations thereof. The organic solvents are used in solutions (14) at concentrations sufficient to loosen the undesired tissue (10) from the connective tissue. By way of example, TBP may be used in the solution in a range of about 0.01% to about 10%. Ethanol and isopropanol may each be in solution (14) in a range of about 0.01% to 100%. However, these organic solvents and ranges are merely exemplary, and those of ordinary skill in the art will understand that the organic solvents and concentrations can be adjusted as necessary to practice various embodiments.

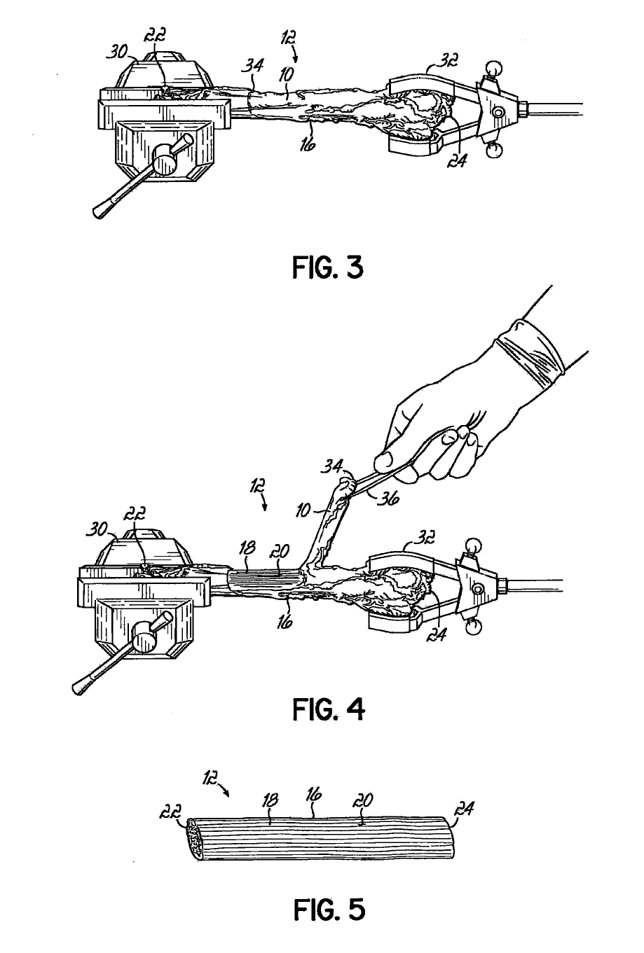

Turning now to FIGS. 3 and 4, in some embodiments, at least one of the first or second opposing ends (22, 24) of the connective tissue, i.e., a tendon (12), is secured prior to stretching. For example, the first opposing end (22) or second opposing end (24) of the connective tissue may be secured by any method known to one skilled in the art such as, for example, by a vice (30), a clamp (32), pliers, a hand, or combinations thereof.

As further illustrated by FIGS. 3 and 4, the connective tissue, i.e., a tendon (12), is stretched prior to peeling away the undesired tissue (10). The connective tissue maybe stretched until it is tight (i.e., a force at least sufficient to remove any slack from the stretched connective tissue), but not so much as to significantly damage it (i.e., less than a force that substantially reduces viability of the connective tissue for transplantation). In one embodiment, the connective tissue is stretched with a force between about 1 Newton and the load of failure (i.e., the load at which the connective tissue breaks). In another embodiment, the connective tissue is stretched with a force between about 1 Newton and about 1000 Newtons. In yet another embodiment, the connective tissue is stretched with a force between about 1 Newton and about 100 Newtons. In a further embodiment, the connective tissue is stretched with a force between about 5 Newtons and about 50 Newtons. The connective tissue may be stretched by any method known to one skilled in the art, such as, for example, by applying a manual pulling force to the opposing ends of the tendon of the connective tissue or by securing the first and second opposing ends (22, 24) of the connective tissue in a device that applies a mechanical pulling force to the opposing ends (22, 24).

The undesired tissue (10) is cut to provide a leading tissue edge (34) that can be grasped so that the undesired tissue (10) may be peeled away from the connective tissue. As further illustrated in FIGS. 3 and 4, in some embodiments the undesired tissue (10) is cut along the width of the intermediate portion (16) between the first (22) and second (24) opposing ends of the connective tissue, i.e., tendon (12), prior to peeling. The undesired tissue is cut to a depth sufficient to transect the undesired tissue (10) layer, but not so deep as to damage the connective tissue. The leading tissue edge (34) may be made at any point along the connective tissue or, in embodiments in which bone, muscle, or organ tissue are still attached to the connective tissue, at any point along the bone, muscle, or organ tissue. Advantageously, the leading tissue edge (34) may be near one of the first (22) or second (24) opposing ends of the connective tissue. The leading tissue edge (34) may be made by any method known to one of ordinary skill in the art such as, for example, with a lifter, scalpel, laser, or any other suitable instrument.

As illustrated in FIGS. 4 and 5, the undesired tissue (10) is peeled away from the tendon (12) leaving the outer surface (20) of the peritendineum (18) of the tendon (12) substantially free of undesired tissue (10). The undesired tissue (10) may be peeled by any method known to one of ordinary skill in the art. By way of example, the undesired tissue (10) may be gripped with fingers; forceps (36), nerve isolators, pliers, or any other suitable instrument, and pulled until the undesired tissue (10) is peeled away from the connective tissue.

Thus, as illustrated in FIG. 5, the methods of the present invention provide a connective tissue, i.e., a tendon (12), having an outer surface (20) of peritendineum (18) that is substantially free of undesired tissue (10). However, in some embodiments of the invention, some undesired tissue may remain attached to or integrated into the connective tissue after the undesired tissue (10) is peeled away. The remaining undesired tissue may be removed from the connective tissue by soaking and/or scrubbing the peeled connective tissue with a cleaning solution. The cleaning solution may include a detergent, an enzyme, a salt solution, an organic solvent, and combinations thereof as known to one having ordinary skill in the art. Any of the detergents, enzymes, salt solutions, and organic solvents described above for use in the soaking solution (14) may be suitable for use in the cleaning solution of the present invention. Examples of suitable detergents include SDS, SDC, Brij-35®, NP-40®, and Nonoxynol-9®, Allowash®, NLS, Tween® 20, and combinations thereof. Examples of suitable enzymes include lipase A, lipase B, trypsin, chymotrypsin, esterase, and combinations thereof. Examples of suitable salt solutions include PBS, Tris solutions, Hepes solutions, NaCl solutions, CaCl₂ solutions, and combinations thereof and may be hypertonic, hypotonic, or isotonic. Examples of suitable solvents include TBP, ethanol, isopropanol, and combinations thereof. It is contemplated that while the cleaning solution may include similar components to the soaking solution (14), the cleaning solution may use additional components or use different ratios of the soaking solution components.

The present invention will be further appreciated in light of the following examples.

### Example 1

Example 1 demonstrates various exemplary conditions that may be used with the methods of the present invention, such as varying the time that the tendon is soaked in solution prior to stripping, as well as varying the components and temperature of the solution.

Porcine patellar tendons, also referred to as bone-tendon-bone (“BTB”), were procured from 6 to 8 month old pigs. The opposing ends of the BTBs were still attached to sections of bone. The BTBs were soaked for 24 hours to 48 hours in various solutions containing enzymes (trypsin, lipase A, or lipase B), detergent (NLS, Allowash®), and/or salt solutions at concentrations described in Table 1.

The various solutions used in this example were made as follows:

Lipase A Solution—Porcine lipase (1000 unit/ml) was added to 25 mM Tris HCI buffer having a pH of 7.8 at 25° C. The Tris HCI Buffer was made by diluting 12.5 ml of 1 M Tris HCl, ph 7.8, in 487.5 ml deionized H₂O (ddH₂O) and then filtered. 1 M Tris HCl was made by dissolving 121 g Tris base with ddH₂O and about 65 ml 12 N HCl to 1 liter and adjusting the pH with HCl or base as needed.

Lipase B Solution—Immediately prior to use, a lipase B stock solution was made by dissolving 20,000 units/ml of porcine pancreas lipase B in cold 5 mM CaCl₂. 5 mM CaCl₂ solution was made by mixing 2.5 ml of 1 M CaCl₂ and 497.5 ml of ddH₂O, then filtering. 1M calcium chloride (CaCl₂) solution was made by dissolving 55.5 g of CaCl₂ was dissolved in 500 ml of ddl H₂O, then sterilized. Reagent B, 3 M sodium chloride (“NaCl”) solution was made by dissolving 175.32 g of NaCl in ddH₂O and sterilizing. Reagent C, 1.5% wt/vol sodium taurochlorate (“auro”) solution was prepared by dissolving 2.25 g taurocholic acid in 250 ml ddH₂O. Reagent D, 75 nM CaCl₂ solution was prepared by mixing 37.5 ml of 1 M CaCl₂ and 462.5 ml of ddH₂O, then filtering. Reagent E, 10 mM sodium hydroxide (“NaOH”), was made by dissolving 400 mg of NaOH in 1000 ml of ddH₂O. The lipase working buffer was prepared by mixing 50 ml of ddH₂O with 20 ml of reagent B, 20 ml of reagent C and 10 ml reagent D. The working buffer was mixed by swirling and the pH was adjusted to 8.0 at 37° C. with reagent E. The lipase B stock solution was diluted in the working solution to 1000 units/ml.

Hypotonic Tris buffer—10 ml of 1M Tris Cl at pH 7.2 was mixed with 0.35 ml of 5% PMSF per liter, 10 ml of 0.5 M EDTA at pH 8.0, 10 ml of 100× penicillin/streptomysin and 970 ml of ddH₂O, 5% PMSF was made by dissolving 2.5 g of PMSF in 50 ml of 200 proof ethanol.

Allowash® solution—Allowash® solution comprises a solution of three detergents: (1) Brij-35® (polyoxyethylene-4-lauryl ether), (2) Nonidet P-40® (octylphenol-ethyleneoxide also known as Tergitol NP-40®) and (3) Nonoxynol-9® (poly(ethylene glycol)p-nonyl-phenyl-ether. A 1× solution of Allowash® contains about 0.066 wt % Brij-35®, about 0.02 wt % NP-40®, and about 0.02 wt % Nonoxynol-9® in endotoxin free water. To make the 1× Allowash® solution 33 ml of 1% Brij® and 1 ml of 10% Igepal® were mixed with 25 ml of 1 M Tris CI at pH 7.2 with 441 ml sterile ddH₂O. The 1% Brij® solution was made by diluting 10 ml of 30% Brij 35® in 290 ml of sterile ddH₂O. The 10% Igepal® (NP-40®) solution was made by mixing 20 ml of Igepal® with 180 ml sterile ddH₂O. To make the 0.1× modified Allowash® solution, 50 ml of 1× modified Allowash® solution was mixed with 25 ml of 1 M Tris HCI at pH 7.2 and 425 ml sterile ddH₂O.

NLS solution—1% (wt/vol) NLS solution was made by dissolving 5 g of NLS in 475 ml of sterile ddH₂O, and 25 ml of Tris HCI at pH 7.2. The 0.1% (wt/vol) NLS solution was made by diluting 50 ml of 1% NLS solution with 25 ml of 1 M Tris HCI and 425 ml sterile ddH₂O.

Trypsin solution—The trypsin solution was made by dissolving 0.25 g of trypsin in 1000 ml of PBS solution (pH 7.4). The trypsin solution is then filtered through a sterile 0.22 μm filter.

Reagents and conditions and procedures.

TABLE 1

| | Step 1 | | | | | Step 3 | | | | |
|---|---|---|---|---|---|---|---|---|---|---|
| | Reagent | Time | Vol | Condition | Step 2 | Reagent | Time | Vol | Condition | Step 4 |
| 1 | Lip A (1000 Unit/ml) | 24 h | 75 ml | 37° C.; 100 rpm | Strip fat | | | | | |
| 2 | Lip A (1000 Unit/ml) | 48 h | 75 ml | 37° C.; 100 rpm | Strip fat | | | | | |
| 3 | Lip B (1000 Unit/ml) | 24 h | 75 ml | 37° C.; 100 rpm | Strip fat | | | | | |
| 4 | Lip B (1000 Unit/ml) | 48 h | 75 ml | 37° C.; 100 rpm | Strip fat | | | | | |
| 5 | 0.25% | 6 h | 75 ml | 37° C.; 100 rpm | Rinse | Lip A | 48 h | 75 ml | 37° C.; 100 rpm | Strip |
| | Trypsin | | | | with IX | (1000 unit/ml) | | | | fat |
| | | | | | PBS | | | | | |
| 6 | 0.25% | 6 h | 75 ml | 37° C.;. 100 rpm | Rinse | LipB | 48 h | 75 ml | 37° C.; 100 rpm | Strip |
| | Trypsin | | | | with 1X | (1000 unit/ml) | | | | fat |
| | | | | | PBS | | | | | |
| 7 | 1% NLS in | 12 h × 2 | 150 ml | RT; 100 rpm | Strip fat | | | | | |
| | 50 mM Tris | | | | | | | | | |
| 8 | 0.1% NLS | 12 h × 2 | 150 ml | RT; 100 rpm | Strip fat | | | | | |
| | in 50 mM | | | | | | | | | |
| | Tris | | | | | | | | | |
| 9 | 0.1X | 12 h × 2 | 150 ml | RT; 100 rpm | Strip fat | | | | | |
| | Allowash ® | | | | | | | | | |
| | in 50 mM | | | | | | | | | |
| | Tris | | | | | | | | | |
| 10 | 1X | 12 h × 2 | 150 ml | RT; 100 rpm | Strip fat | | | | | |
| | Allowash ® | | | | | | | | | |
| | in 50 mM | | | | | | | | | |
| | Tris | | | | | | | | | |

### Example 2

Porcine patellar tendon, BTB, was procured from 6 to 8 month-old pigs. The opposing ends of the BTB were still attached to sections of bone. The BTB was soaked for 24 hours in solutions containing enzymes (trypsin, lipase A, or lipase B), detergents (Triton® X-100, NLD, Allowash®), and/or salt solutions (Tris or phosphate buffered saline (“PBS”)) at concentrations described in Table 2. Each condition was test using three BTBs.

The solutions were made as described in Example 1 or as known by one of ordinary skill in the art. The volume of the solution for each condition was 150 ml per BTB. Groups 4-11 were placed on a shaker at 100 RPM.

Difficulty level is defined as the strength needed to peel tissue from the tendon. The most difficult level possible is 6 and the easiest level possible is 1.

Table 2 illustrates the results of this study. It should be noted that the methods of the present invention generally decreased both 1) the difficulty level and 2) the time required to remove the undesired tissue from the ligament. At the same time, the methods decrease the likelihood of damaging the tendon.

TABLE 2

| | | Difficulty | Time | |
|---|---|---|---|---|
| Group # | Processing | Level | (Min.) | Comments |
| 1 | Fresh (strip with scalpel) | 4 | 6-20 | (1) Easy to damage tendon and |
| | | | | hard to completely remove |
| | | | | undesired tissue. |
| | | | | (2) Requires skill and patience. |
| 2 | Fresh (strip with lifter) | 5 | 5 | (1) Undesired tissue can be |
| 3 | PBS, 4° C. @24 h | 6 | 4 | peeled away as an intact sheet. |
| 4 | PBS RT, 24 h | 4 | 2 | (2) Tendon is not damaged |
| 5 | PBS 37° C., 24 h | 4 | 2 | with good initial cut of |
| | | | | undesired tissue. |
| | | | | (3) Strength is needed to peel |
| | | | | the undesired tissue away, |
| | | | | especially with groups 2 and 3. |
| 6 | 1% Triton ® X-100, RT, 24 h | 3 | 1-1.5 | (1) Undesired tissue can be |
| 7 | 0.1X Allowash ® RT, 24 h | 3.5 | 1-1.5 | peeled away as an intact sheet. |
| 8 | IX Allowash ® RT, 24 h | 3 | 1-1.5 | (2) Easy to make a clear initial |
| 9 | 0.1% NLS RT, 24 h | 3 | 1-1.5 | cut of undesired tissue. |
| | | | | (3) Easy to peel undesired |
| | | | | tissue away. |
| | | | | (4) Slightly easier to damage |
| | | | | tendon in group 7 compared to |
| | | | | groups 6, 8, and 9. |
| 10 | Lip A, 1000 U/ml37 C, 24 h | 4 | 3-4 | (1) Hard to make a clear initial |
| 11 | 0.25% Trypsin, 37° C., 6 h, | 4 | 3-4 | cut b/c undesired tissue is |
| | then Lip A, 1000 U/ml 37° C., | | | looser. |
| | 24 h | | | (2) Tissue can not be peeled |
| | | | | away as an intact sheet and it |
| | | | | takes longer to remove. |

As various changes could be made in the above-described aspects and exemplary embodiments without departing from the scope of the invention, it is intended that all matter contained in the above description shall be interpreted as illustrative and not in a limiting sense. To that end, while the Figures depict and the detailed description discusses a tendon, those skilled in the art will recognize that a tendon is merely exemplary and that the methods are applicable to other biological components that require tissue removal such as, for example, ligaments.

1. A method of removing undesired tissue from a connective tissue comprising: stretching a connective tissue, the connective tissue having an intermediate portion extending between first and second opposing ends of the connective tissue; and peeling undesired tissue from the stretched connective tissue.

2. The method of claim 1 wherein the connective tissue is chosen from a tendon, a ligament, and a meniscus.

3. The method of claim 1 further comprising excising the connective tissue from a subject.

4. The method of claim 1, further comprising soaking the connective tissue in a solution prior to stretching the connective tissue.

5. The method of claim 4 wherein the connective tissue is soaked in the solution for at least 0.5 hours.

6. The method of claim 4 wherein the solution is at a temperature in a range of about 4° C. to about 42° C.

7. The method of claim 4 wherein the solution includes one or more components chosen from a detergent, an enzyme, a salt solution, an organic solvent, and combinations thereof.

8. The method of claim 7 wherein the detergent is chosen from sodium dodecyl sulfate (SDS), sodium deoxycholate (“SDC”), t-Octylphenoxypolyethoxyethanol (“Triton® X-100”), polyoxyethylene-4-lauryl ether (“Brij-35”), octylphenol-ethyleneoxide (“NP-40®”), poly(ethylene glycoljp-nonyl-phenyl-ether (“Nonoxynol-9®”), polyoxyethylenesorbitan (“Tween® 20”), n-lauroyl sarcosinate (“NLS”), and combinations thereof.

9. The method of claim 7 wherein the enzyme is chosen from lipase A, lipase B, esterase, trypsin, chymotrypsin, esterase, and combinations thereof.

10. The method of claim 7 wherein the salt solution is chosen from phosphate buffered saline, Tris solution, Hepes solution, NaCl solutions, CaCl₂ solutions, and combinations thereof.

11. The method of claim 7 wherein the salt solution is one of isotonic, hypotonic, or hypertonic.

12. The method of claim 7 wherein the organic solvent is chosen from tributyl phosphate (“TBP”), ethanol, isopropanol, and combinations thereof.

13. The method of claim 1, further comprising securing at least one of the first or second opposing ends prior to stretching the intermediate portion.

14. The method of claim 13 wherein the first opposing end and/or second opposing end is secured by a vice, a clamp, pliers, a hand, or combinations thereof.

15. The method of claim 1, further comprising cutting the undesired tissue along the width of the connective tissue, prior to peeling away the undesired tissue.

16. The method of claim 1, further comprising soaking and/or scrubbing the connective tissue with a cleaning solution to remove any undesired tissue that remains attached to the connective tissue after peeling the undesired tissue.

17. The method of claim 16 wherein the cleaning solution includes one or more components chosen from a detergent, an enzyme, a salt solution, an organic solvent, and combinations thereof.

18. The method of claim 17 wherein the detergent is chosen from SDS, SDC, Triton® X-100, Brij-35®, NP-40®, Nonoxynol-9®, Tween® 20, NLS, and combinations thereof.

19. The method of claim 17 wherein the enzyme is chosen from lipase A, lipase B, esterase, trypsin, chymotrypsin, and combinations thereof.

20. The method of claim 17 wherein the salt solution is chosen from phosphate buffered saline, Tris solution, Hepes solution, NaCl solutions, CaCl₂ solutions, and combinations thereof.

21. The method of claim 17 wherein the salt solution is one of isotonic, hypotonic, or hypertonic.

22. The method of claim 17 wherein the organic solvent is chosen from TBP, ethanol, isopropanol and combinations thereof.

23. A method of removing undesired tissue from a connective tissue comprising: excising a connective tissue from a subject wherein the connective tissue has an intermediate portion extending between first and second opposing ends; soaking the connective tissue in a solution; securing the at least one of the first and second opposing ends of the connective tissue; stretching the intermediate portion of the connective tissue; cutting the undesired tissue along the width of the connective tissue; and peeling the undesired tissue away from the intermediate portion of the connective tissue.

24. The method of claim 23 wherein the connective tissue is chosen from a tendon, a ligament, and a meniscus.

25. The method of claim 23 wherein the solution is in a temperature range of about 4° C. to about 40° C.

26. The method of claim 23 wherein the connective tissue is soaked in the solution for at least 0.5 hours.

27. The method of claim 23 wherein the solution includes one or more components chosen from a detergent, an enzyme, a salt solution, an organic solvent, and combinations thereof.

28. The method of claim 27 wherein the detergent is chosen from SDS, SDC, TBP, Triton® X-100, Brij-35®, NP-40®, Nonoxynol-9®, NLD, Tween® 20, NLS, and combinations thereof; the enzyme is chosen from lipase A, lipase B, esterase, trypsin, chymotrypsin, and combinations thereof; the salt solution is chosen from phosphate buffered saline, Tris solution, Hepes solution, NaCl solutions, CaCl₂ solutions and combinations thereof; and the organic solvent is chosen from TBP, ethanol, and isopropanol.

29. The method of claim 27 wherein the salt solution is one of isotonic, hypotonic, or hypertonic.

30. The method of claim 23, further comprising soaking and/or scrubbing the connective tissue with a cleaning solution to remove any undesired tissue that remains attached to the tendon or ligament after peeling the tissue.

31. The method of claim 30 wherein the cleaning solution includes one or more components chosen from a detergent, an enzyme, a salt solution, an organic solvent, and combinations thereof.

32. The method of claim 30 wherein the detergent is chosen from SDS, SDC, TBP, Triton® X-100, Brij-35®, NP-40®, Nonoxynol-9®, Tween® 20, NLS, and combinations thereof; the enzyme is chosen from lipase A, lipase B, esterase, trypsin, chymotrypsin, and combinations thereof; the salt solution is chosen from phosphate buffered saline, Tris solution, Hepes solution, NaCl solutions, CaCl₂ solutions and combinations thereof; and the organic solvent is chosen from TBP, ethanol, and isopropanol.

33. The method of claim 31 wherein the salt solution is one of isotonic, hypotonic, or hypertonic.
